(19) United States
(12) Patent Application Publication
(10) Pub. No.: US 2009/0198326 A1
(43) Pub. Date: Aug. 6, 2009

(54) ACCOMMODATIVE INTRAOCULAR LENS SYSTEM

(75) Inventors: Stephen Q. Zhou, Irvine, CA (US); Christopher D. Wilcox, Mission Viejo, CA (US)

Correspondence Address: FROST BROWN TODD, LLC, 2200 PNC CENTER, 201 E. FIFTH STREET, CINCINNATI, OH, 45202, US

(73) Assignee: MEDENNIUM INC., Irvine, CA (US)

(21) Appl. No.: 12/023,493

(22) Filed: Jan. 31, 2008

(51) Int. Cl. A61F 2/16; A61B 19/00

(52) U.S. Cl. 623/6.34; 623/6.32; 623/6.11; 128/898

(57) ABSTRACT

An accommodative intraocular lens system for treating presbyopic is disclosed. The system includes a first lens having negative optic power adapted for placement in the posterior chamber of the eye and capable of moving forward and back along the optic axis; and a second lens having a positive optic power which is implanted within the capsular bag. The second lens can be the natural crystalline lens of the eye. The position of the first lens, forward or back relative to the second lens, focuses the eye for seeing distant or close-in objects.

### BACKGROUND OF THE INVENTION

This invention relates to the field of intraocular lenses (IOLs) which can provide accommodation to enable a patient to see both distant and near objects.

Presbyopia is part of the natural ageing process which happens to everyone around the age of 50. In a presbyopic patient, the natural human crystalline lens loses its ability to change from a thin to a thick lens, resulting in the loss of near vision. The natural human crystalline lens can also become less transparent and block light from reaching the retina; in such a case, the cloudy natural crystalline lens, or the cataract lens, can be surgically removed and be replaced with an artificial lens, or intraocular lens. Since the majority of cataract patients are over 50 years of age, it would be an ideal situation if an intraocular lens could not only replace the cataract lens, but also provide the cataract patient with accommodation, i.e., allowing the patient to see both distant or near objects as the situation requires.

There are approximately 2 million cataract procedures performed annually in the US. Since presbyopia affects to the entire senior population over 50 years old, there have been tremendous efforts in the scientific and industrial field to find a solution for restoring accommodation. Based on the multiplicity of the optics involved in the design, accommodative IOLs can be divided into two groups: mono-optic design and bi-optic design.

Mono-optic design utilizes the optic shift along the optical axis of the eye to provide a small amount of lens diopter shift corresponding to the lens power for near vision (lens shifted forward toward cornea) and distance vision (lens shifted backward toward retina). FIG. 1 demonstrates the optic's shift along the optical axis from prior art literature. The distance of the optic shift is generally believed to be about 1 mm, which correlates to about 1 diopter change. Examples of similar mono-optic designs can be found in U.S. Pat. No. 6,176,878 (Gwon, et al, issued Jan. 23, 2001); U.S. Pat. No. 6,387,126 (Cumming, issued May 14, 2002); U.S. Pat. No. 6,485,516 (Boehm, issued Nov. 26, 2002); U.S. Pat. No. 6,406,494 (Laguette, et al, issued Jun. 18, 2002); U.S. Pat. No. 6,524,340 (Israel, issued Feb. 25, 2003); and U.S. Pat. No. 6,533,813 (Lin, et al, issued Mar. 18, 2003), all of which are incorporated by reference herein.

For a generally acceptable accommodative lens, a minimal diopter change of 2 diopters is required with an ideal value of 3 diopters shift. In order to increase the effectiveness of the optic shift of about 1 mm inside the capsular bag along the optical axis, a bi-optic design has been reported in the US patent literature. FIG. 2 is a typical example of such a prior art bi-optic design. In FIG. 2, the negative lens is coupled with a positive lens. The separation of about 1 mm between the 2 optics will correlate to about 2 diopters of the total lenses power change. This is obviously a significant improvement over the mono-optic accommodative lens design described above.

The majority of US patent literature in the area of the bi-optic accommodative lens design share a common structural feature: two optics coupled with a connection means which separates and controls the distance between the two optics. Examples of such designs can be found in U.S. Pat. No. 6,423,094 (Sarfarazi, issued Jul. 23, 2002); U.S. Pat. No. 6,231,603 (Lang, et al, issued May 15, 2001); U.S. Pat. No. 6,464,725 (Skotton, issued Oct. 15, 2002); U.S. Pat. No. 6,818,158 (Pham, et al, issued Nov. 16, 2004); and U.S. Pat. No. 7,223,288 (Zhang, et al, issued May 29, 2007), all of which are incorporated by reference herein.

In addition to the bi-optics connected to each other, there are alternative bi-optic designs which comprise two separate optics, not connected directly each other. For example, U.S. Pat. No. 6,616,692 (Glick, et al, issued Sep. 9, 2003) discloses an intraocular lens combination comprising a first optic, second optic, and movement assembly. The first optic has a negative optic power and is adapted to be placed in a substantially fixed position in a mammalian eye. The second optic has a higher optical power than the first optic. The movement assembly is coupled to the second optic and is adapted to cooperate with the eye, for example, the zonules, ciliary muscle and capsular bag of the eye, to effectively accommodate movement of the second optic in the eye (FIG. 3). As can be seen from FIG. 3, the plus lens inside the capsular bag provides the movement from the substantially fixed negative lens which is outside the capsular bag. The distance, which the moving plus lens can provide, is limited by the capsular bag's dimension. As in the mono-optic case, the movement distance is generally believed to be about 1 mm. In a much similar fashion, U.S. Pat. No. 6,767,363 (Bandhauer, et al, issued Jul. 27, 2004) and U.S. Pat. No. 7,150,760 (Zhang, issued Dec. 9, 2006) independently disclose a lens combination wherein the first optic with a negative power is fixed in the anterior chamber of the eye while the second optic with a positive power is located inside the capsular bag and is designed as such that it moves along the optic axis to cause the distance change relative to the first negative lens, thus providing the eye with accommodation (see FIG. 4). In all of these three inventions, the negative lens is fixed either in the posterior chamber or in the anterior chamber. It is the positive lens, which is located inside the capsular bag, that provides the movement along the optical axis.

US Published Patent Application 2005/0107873, Zhou, published May 19, 2005, describes a full-size accommodative intra-ocular lens which is implanted within the capsular bag and which moves between a first and a second diopter power based on the movement of the capsule.

In summary, it is generally accepted by ophthalmologists that the lens inside the capsular bag can shift along the optic axis about 1 mm which corresponds to about 1 diopter of power change in a mono-optic lens design and to about 2 diopters of power shift in a bi-optic design lens system.

It would be useful to have an alternative design for the bi-optic lens system comprising a negative and a positive optic to provide an effective accommodation by a large separation between the 2 optics. The bi-optic lens system of the present invention is designed to do just that.

### BRIEF SUMMARY OF THE INVENTION

An object of the present invention is, therefore, to provide an effective accommodation for the eye by increasing the difference in distance between a moving first optic and a second optic of a bi-optic lens system. The first optic in the system is a negative lens positioned substantially in the posterior chamber. Unlike the bi-optic system in the prior art, wherein the first lens is substantially fixed in a position, the first optic of the present invention is not substantially fixed in a position, rather it shifts along the optical axis to provide a mechanism for changing the distance between the first optic and the second optic. The second optic of the present invention is a positive lens positioned inside of the capsular bag of the eye. Preferably, the second optic provides an additional mechanism for shifting along the optic axis.

The present invention can also provide a safe and biocompatible lens system that can be easily implanting through a small incision.

Still another object of the present invention is to provide an anatomic structure for both the first optic and the second optic so that a maximum change in distance between the first optic and the second optic can be achieved.

These and other advantages of the bi-optic design of the present invention can be understood and will become apparent to those who are skilled in the art from the following drawings and the detailed descriptions.

### BRIEF DESCRIPTION OF THE DRAWINGS

FIG. 1 is a prior art mono-optic accommodative lens design which shows the optic body shift along the optical axis from the posterior position (FIG. 1A) towards the anterior chamber (FIG. 1B).

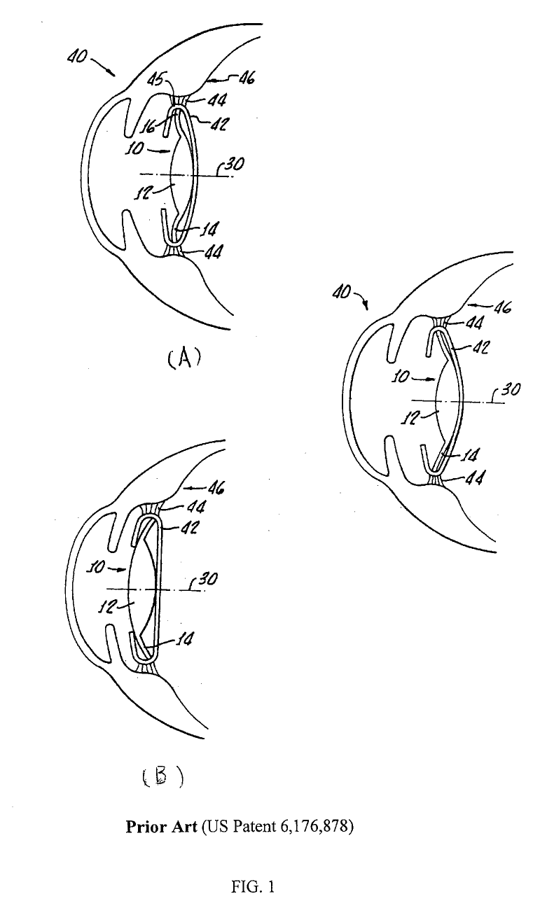

FIG. 2 is a prior art bi-optic accommodative design wherein the distance between the negative lens and the positive lens can change up to about one mm, corresponding to about 2 diopter of change.

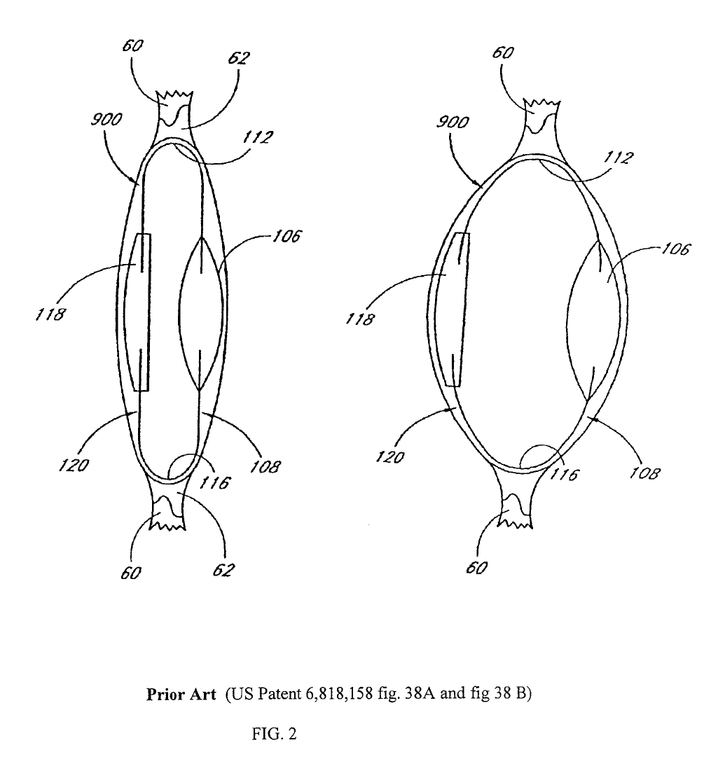

FIG. 3 is another prior art bi-optic design wherein the negative lens is substantially fixed in the posterior chamber and the positive lens inside the capsular bag shifts along the optical axis, in a similar manner as in FIG. 1, to provide for various distances between the two optics.

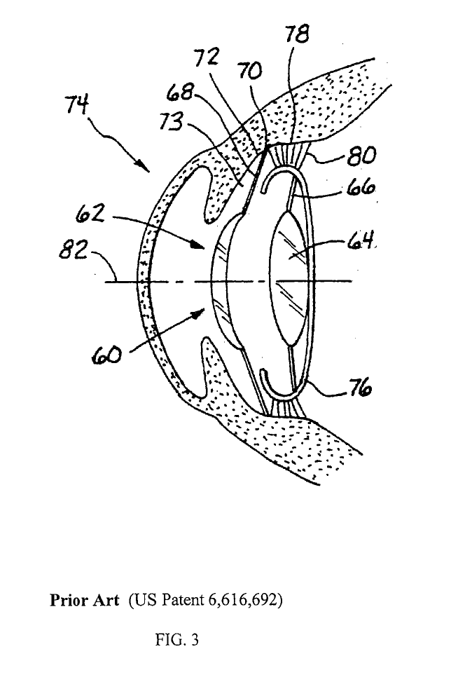

FIG. 4 is still another prior art bi-optic design wherein the negative lens is fixed in the anterior chamber and the positive lens inside the capsular bag shifts along the optical axis in a similar manner as in FIG. 1, to provide a mechanism for accommodation.

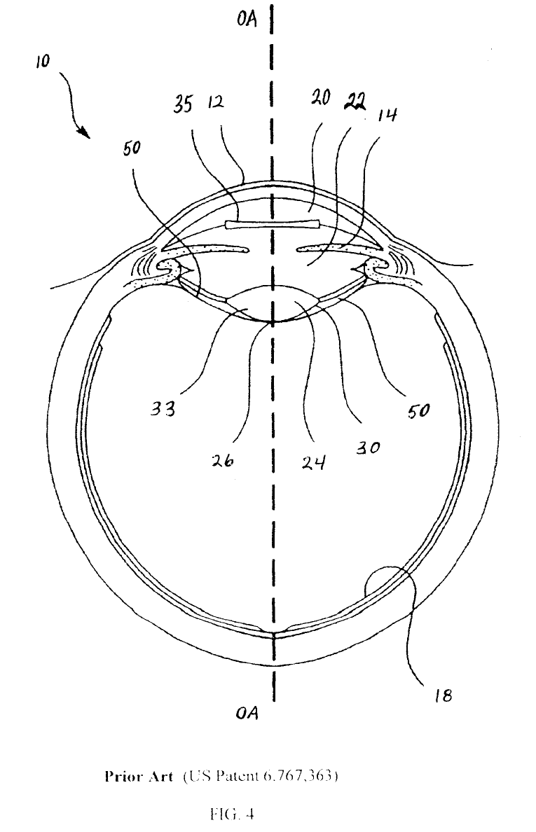

FIG. 5 shows a schematic representation of the bi-optic accommodative lens system of the present invention wherein the first optic 10 with a negative power is substantially located in the posterior chamber of the eye and the second optic 11 with a positive power and full-sized IOL design is positioned inside the capsular bag 2 of the eye. The first optic 10 is not fixed in a specific position, rather it moves along the optical axis 1. As a result, the distance between the two optics changes, which in turn results in a change in the overall power of the bi-optic system.

FIG. 6 is another embodiment of the present invention wherein the first lens 20 provides the shift along the optic axis 1, while the second positive lens 21 with an optic body of about 6 mm diameter is fixed inside the capsular bag 2.

FIG. 7 is still another embodiment of the present invention wherein the first lens 30 provides the shift along the optic axis 1, while the second lens 31 with a positive optic provides additional mechanism for shifting along the optical axis 1.

FIG. 8 is an embodiment of the first lens (used in the present invention) with a negative optic which has a plate haptic structure for positioning the optic substantially in the posterior chamber and the haptic bodies are structured such that the lens is not permanently fixed in a specific position, rather it moves in the aqueous humor of an eye along the optic axis. Optionally, there is a small hole in the center of the optic body as shown in FIG. 8(B). The posterior surface of the optic body can have the substantially same curvature as that of the haptic body (FIG. 8(C)) or a different curvature as shown in FIG. 8(D).

FIG. 9 is another embodiment for the first lens (which can be used in the present invention) with a negative optic power structure whereto are attached multiple piece haptics.

FIG. 10 is still another embodiment for the first lens (which can be used in the present invention) with a negative optic power to which are attached two haptics.

FIG. 11 is a further embodiment of the first lens design.

FIG. 12 is additional embodiment of the first lens design wherein the posterior surface of the haptic has substantially the same curvature as the posterior surface of the optic body to form a continuous posterior surface as shown in FIG. 12B. Of course, it is also possible for the haptics to take a different curvature from the curvature of the posterior surface of the optic body, as shown in FIG. 12(C).

FIG. 13 is an alternative design for the design shown in FIG. 12.

FIG. 14 is a further additional embodiment of the first lens design (which can be used in the present invention) wherein the haptic bodies are extended from the optic body.

### DETAILED DESCRIPTION OF THE INVENTION

“Anterior Chamber” is an anatomic term which defines the fluid-filled space between the iris and the innermost corneal surface (endothelium) of an eye. Anterior chamber depth is the distance between the iris and inner surface of the cornea typically in the range of about 2 mm to about 4 mm.

“Posterior Chamber” is another anatomic term which defines the space between the back of iris and the front face of vitreous of an eye.

“Full-sized IOL” is defined as an IOL with its optic body mimicking the natural crystalline lens, usually with its optic diameter in the range of 8 to 10 mm, preferably about 9 mm, while the central lens thickness is in the range of 2 to 5 mm, preferably about 3.5 mm.

“Phakic” means that the natural crystalline lens is still present in the eye. For example, a phakic IOL means an IOL which works together with the intact natural crystalline lens to correct refractive errors.

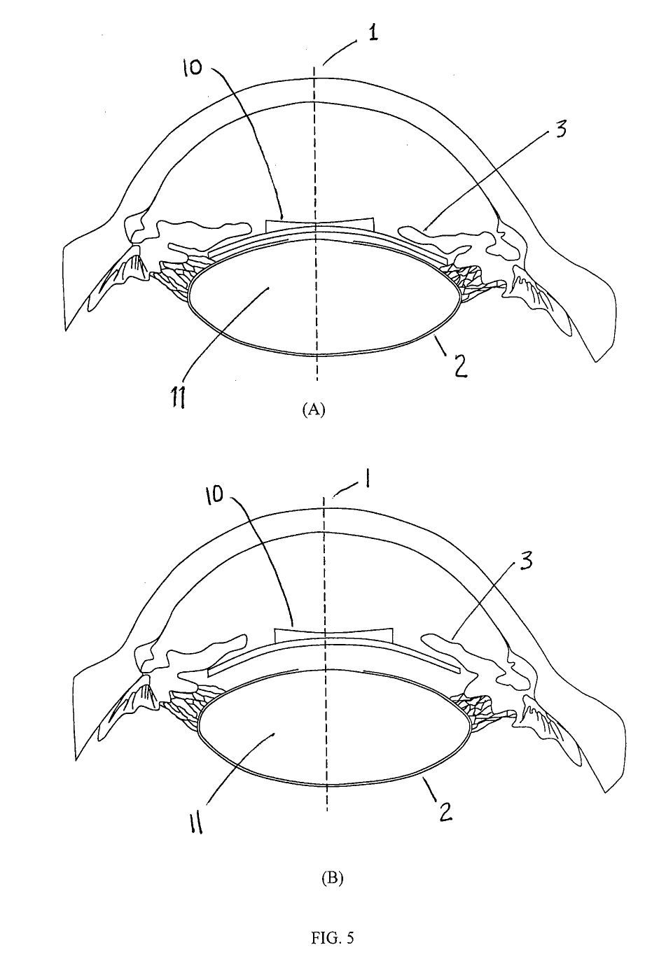

In one of the preferred embodiments of the present invention shown in FIG. 5, there are two lenses in the bi-optic accommodative lens system. The first lens 10 consists of an optic body with a negative power and multiple haptic bodies extended from the optic body which is substantially located in the posterior chamber of an eye. The first lens is structured as such that once it is positioned in the posterior chamber, it provides no permanent fixation in a specific position. Rather, it is configured to shift along optical axis 1 in response to the zonule's muscle movement. Because the anterior chamber is a large empty space, it provides room for the first lens to shift anteriorly as it is shown in FIG. 5(B). The second lens 11 is located inside the capsular bag 2 of the eye. It has a positive optic with a fall-sized lens design. The posterior surface of the first lens 10 is preferably a concave structure with its curvature similar to that of the anterior convex surface of the second lens 11. This way, when the two lenses move close together, these two surfaces are in almost complete contact each other, as shown in FIGS. 5(A) and (B), so that L (the distance between two lenses) trends to zero.

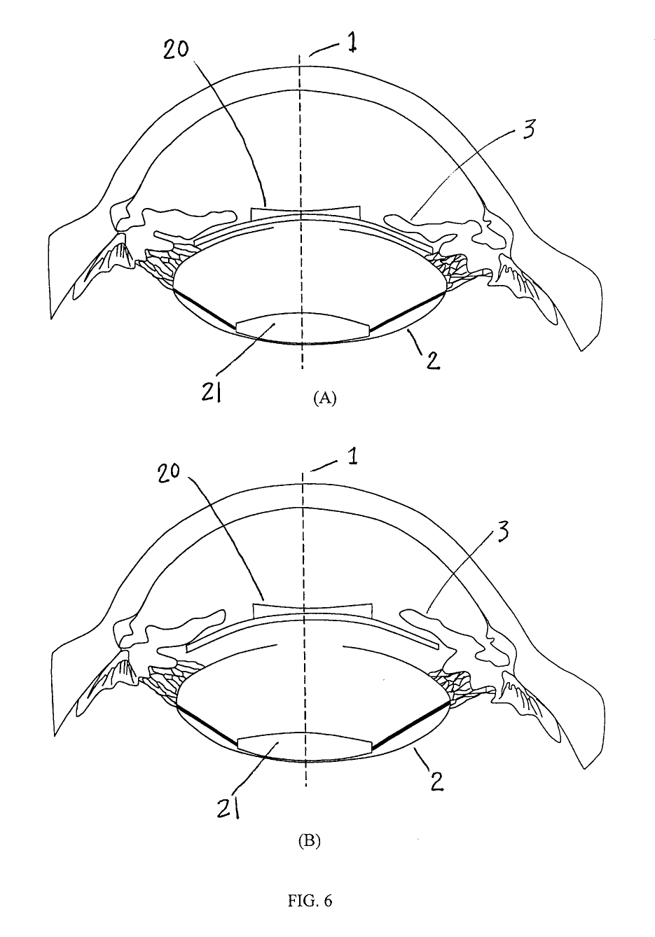

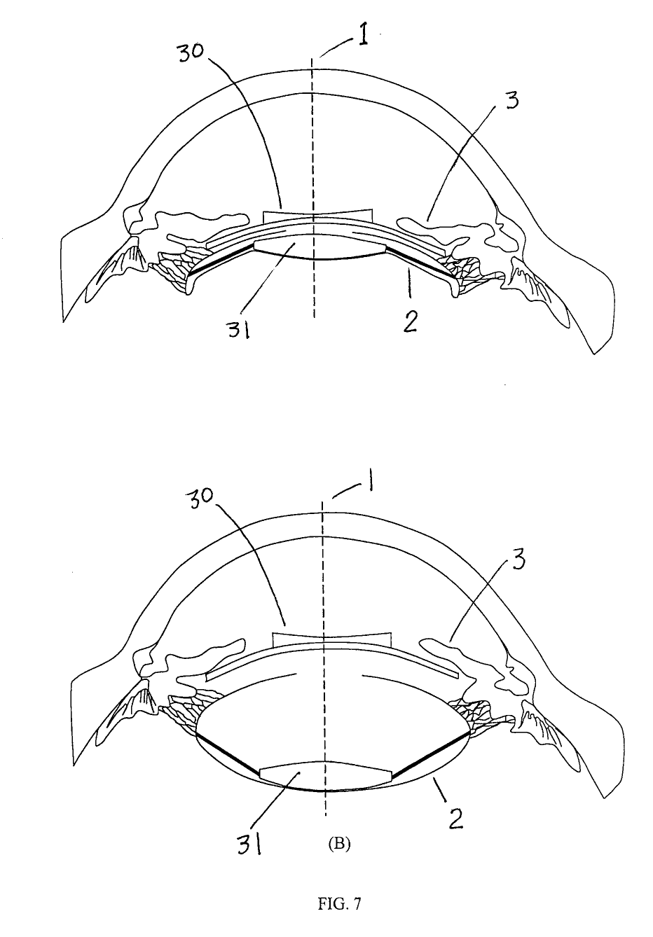

The second lens inside the capsular bag can have various designs suitable for the present invention. For example, it can be a full-sized lens design (FIG. 5). It can also be a 3 piece lens design (FIG. 6). It can still be some other special design for providing additional mechanism for shifting the second optic body along the optical axis (FIG. 7). When this second lens movement is coupled with the first lens shift, it provides the maximum distance variations between the first optic and the second optic. This maximum change in distance between the two optics provide a maximum level of accommodation, as explained in the following optic equation.

When the first negative lens is coupled with the second positive lens, the total focus power of the bi-optic system is dependent on the lens power of each individual lens and the distance between them, as defined by the following theoretical bi-optical component equation.

Dₜₒₜₐₗ=D₁+D₂−(D₁×D₂×L)

Where Dₜₒₜₐₗ is the total power (in diopters) of the bi-optic system
D₁ is the power (in diopters) of the first optic, preferably negative power
D₂ is the power (in diopters) of the second optic, preferably positive power
L is the distance (in meters) between the two optical components

While other factors also contribute to the total diopter power of the bi-optic system, such as relative distance of each individual lens to the retina, the above optics equation applies to the bi-optic system of the present invention. For example, when the negative first lens of the present invention is −10 diopter while the second lens is +30 diopter, if L is zero, then Dₜₒₜₐₗ is +20; if L is 1 mm, then Dₜₒₜₐₗ is 20.3 diopter; if L is 2 mm, then Dₜₒₜₐₗ is 20.6. So it is easily understood that the larger the L, the higher the diopter of bi-optic lens system. It is also important to understand from the above equation for the same L, the larger the optic diopter of the negative lens D₁ and the positive lens D₂, the larger the accommodation power of the bi-optic system. This is because ΔD=D₁×D₂×L, wherein AD is the accommodation power of the bi-optic lens system.

In order to achieve the design objectives of the present invention, the first negative lens has to be able to move anteriorly (toward the cornea) and posteriorly (toward the retina) along the optical axis. It is known that aqueous humor flows from the posterior chamber through the pupil to the anterior chamber at about 2 μl/minute. Aqueous humor is generated from the ciliary body muscles which is also the origin of the accommodation process. In a non-accommodative situation, zonules pull the natural lens to the thin central lens thickness which allows the eye to see a distant object. When the eye accommodates, ciliary muscle contracts to relax the zonules to allow the natural lens to assume a more spherical shape (or a thick lens shape) to focus on a near object, such as reading. Meanwhile, there is aqueous humor outflow from the posterior chamber, via the pupil to the anterior chamber. This outflow is the driving force for the negative lens of the present invention to move anteriorly by separating the negative lens further away from the positive lens. Because of the empty space provided by the anterior chamber, it allows the first lens of the present invention to shift anteriorly. For this reason, the negative lens cannot be fixed in any permanent way to hinder such an anterior shift. In order to understand this non-permanent fixation principle, the following examples are used for the purpose of demonstration, but not to limit the scope for the design features of the negative lens.

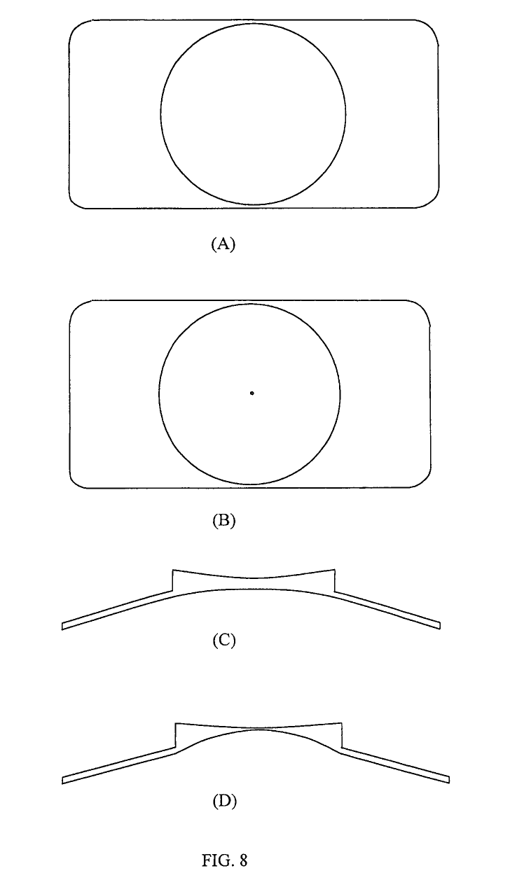

The first example for the first lens of the present invention is shown in FIG. 8. The lens has an optic body with multiple haptic bodies extending from the optic body. The overall length of the lens (diagonal distance from one end of the haptic body to the opposite side of the haptic body) is about 12 mm or shorter to ensure the lens is not oversized in length. The haptic body is very thin to keep the optic body centrally located within the pupil. It is optional that the posterior surface of the first lens be a concave surface (see FIG. 8C) with its curvature similar to the anterior convex surface of the second lens. This way, when the first lens moves towards the second lens, the convex anterior surface of the second lens situates itself into the concave surface of the first lens to the maximal extent, so that the distance between them is minimal (L trends toward zero, such as shown in FIG. 5A and FIG. 7A) to achieve the lowest total diopter power of the bi-optic system. Optionally, at the center of the optic body there can be a hole with a diameter in the range of 50-100 μm to allow the aqueous humor to flow through (see FIG. 8B). In order to not cause an intraocular pressure increase after the surgery, it is essential for the aqueous humor to continuously flow from the posterior chamber to the anterior chamber either through the hole at center of the first lens or through a combination of the central hole and the channel created by surgical iridectomy or laser iridectomy.

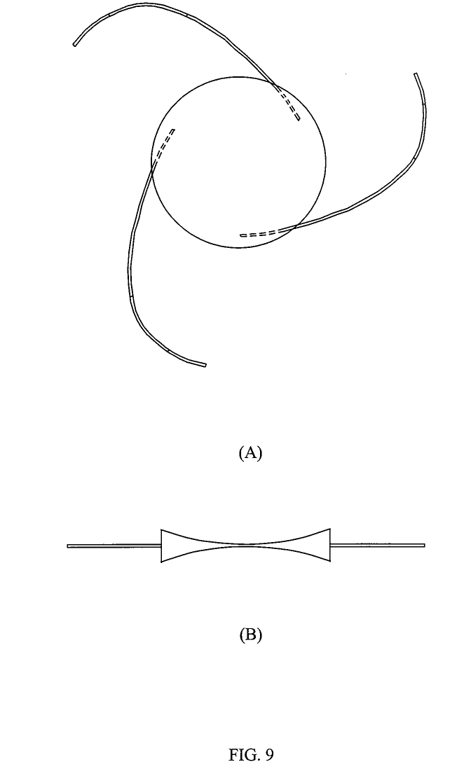

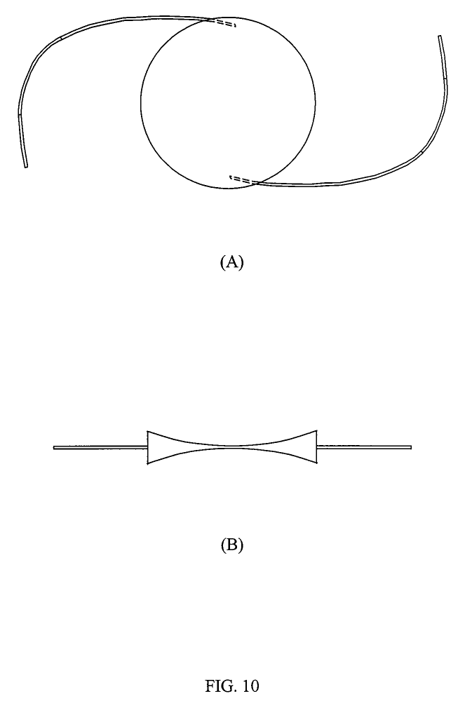

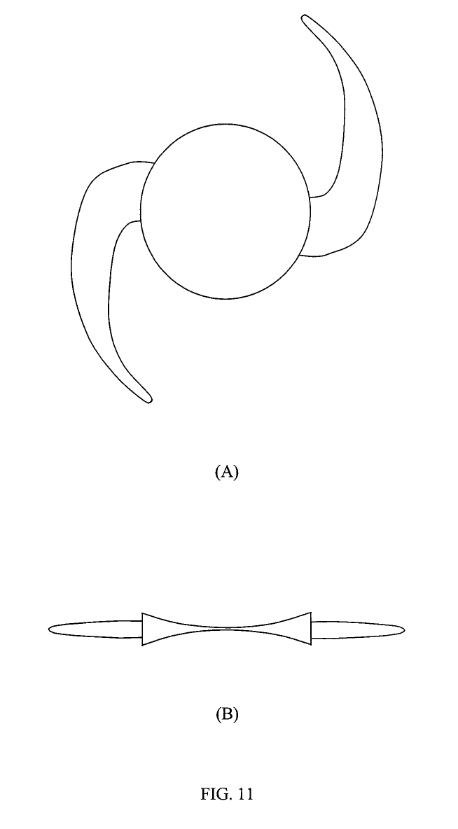

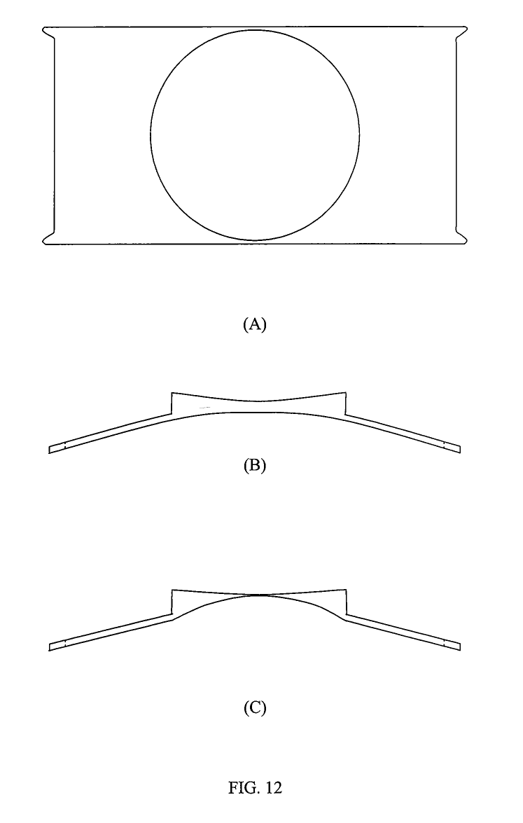

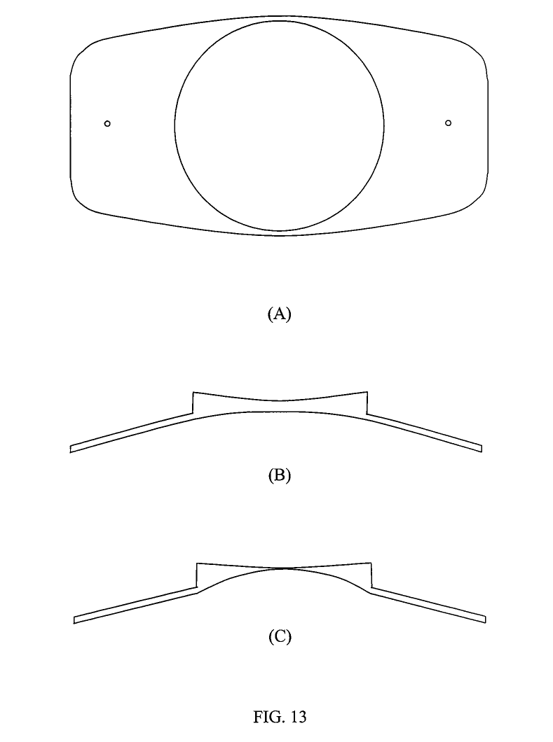

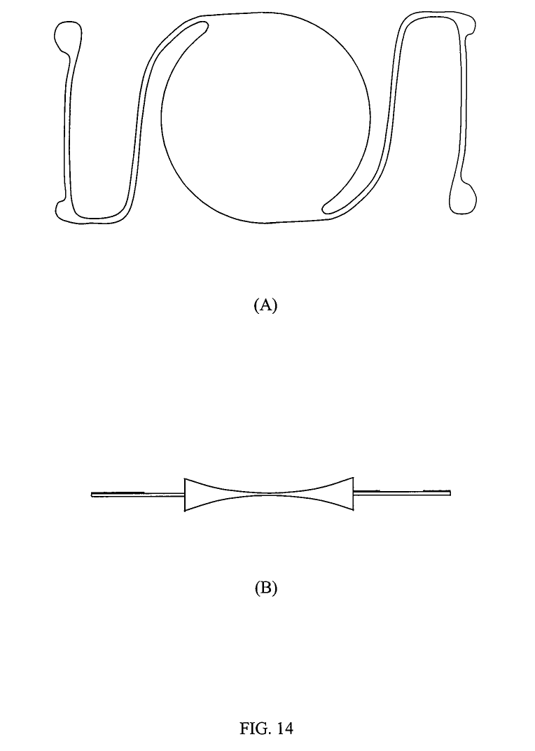

Other examples of the first lens of the present invention are demonstrated in FIGS. 9-14. The common features of these examples are: a lens with a negative optic body, preferably with a power diopter of −5 diopter or higher (such as −10 diopter), and a lens with an overall length of about 12 mm or less so that the lens is not permanently fixed but rather floats in the aqueous humor with the optic body substantially in the posterior chamber. As the aqueous humor flows from the posterior chamber to the anterior chamber, the optic body can shift along the optic axis. In addition, the optic body can optionally have a small hole with a diameter in the range 50-100 μm to allow the aqueous humor to flow through.

Exemplary designs for the second lens inside the capsular bag include but are not limited to: a full-sized IOL design shown in FIG. 5, a 3 piece IOL design shown in FIG. 6, and a special design with features for guiding the optic body shifting inside the capsular bag along the optic axis (FIG. 7). As matter of fact, all mono-optic accommodative lens designs which can be implanted inside the capsular bag are suitable as the second lens of the bi-optic lens system of the present invention. Examples of the mono-optic accommodative lens designs can be found in U.S. Pat. No. 6,176,878 (Gwon, et al, issued Jan. 23, 2001); U.S. Pat. No. 6,387,126 (Cumming, issued May 14, 2002); U.S. Pat. No. 6,485,516 (Boehm, issued Nov. 26, 2002); U.S. Pat. No. 6,406,494 (Laguette, et al, issued Jun. 18, 2002); U.S. Pat. No. 6,524,340 (Israel, issued Feb. 25, 2003); and U.S. Pat. No. 6,533,813 (Lin, et al, issued Mar. 18, 2003), all incorporated by reference herein.

It is particularly interesting to point out that in FIG. 5 the second lens is a full-sized IOL which mimics the human natural crystalline lens. In a similar scenario wherein the human natural crystalline lens serves as the second lens of the bi-optic accommodative lens system of the present invention, the first lens becomes a phakic IOL. Although phakic IOLs have been extensively patented (for example, in U.S. Pat. No. 6,428,574 (Valunin, et al, issued Aug. 6, 2002); U.S. Pat. No. 6,015,435 (Valunin, et al, issued Jan. 18, 2000); U.S. Pat. No. 6,506,212 (Zhou, et al, issued Jan. 14, 2003); U.S. Pat. No. 5,913,898 (Feingold, issued Jun. 22, 1999); and U.S. Pat. No. 5,766,245 (Fedorov, et al, issued Jun. 16, 1998), all incorporated by reference herein) for effectively correcting various refractive errors, such as severe myopia and severe hyperopia, using a specially designed first lens which can shift its position along the optic axis is novel. In this sense, using only the first negative lens in combination with the human natural crystalline lens of a presbyopic patient for achieving distant vision (such as driving) and near vision (such as reading) is within the scope of the present invention.

What is claimed is:

1. An accommodative lens system, comprising:
(a) a first lens with a negatively powered optic body adapted to be located substantially in the posterior chamber of a human eye; said first lens configured so as not to be fixed in a specific position along the optic axis, but rather to remain in the central position in the pupil and to shift along the optic axis; and
(b) a second lens with a positively powered optic body configured to be implanted inside the capsular bag.

2. The accommodative lens system of claim 1 wherein said first lens is configured so as to permit the flow of aqueous humor past it, as said lens shifts positions.

3. The accommodative lens system of claim 2 wherein said first lens is a plate haptic body design.

4. The accommodative lens system of claim 3 wherein said plate haptic body has a thickness in the range of about 50 to about 200 μm, and wherein the overall length of said first lens is approximately 12 mm or less.

5. The accommodative lens system of claim 2 wherein said first lens is a multiple piece lens design wherein two or more haptic bodies are attached to and extended from the central optic body.

6. The accommodative lens system of claim 2 wherein said second lens is a full-sized IOL.

7. The accommodative lens system of claim 2 wherein said second lens is a plate haptic body design.

8. The accommodative lens system of claim 2 wherein said second lens is a multiple piece lens design wherein two or more haptic bodies are attached to and extended from the central optic body.

9. The accommodative lens system of claim 2 wherein said second lens is configured to allow its optic body to shift along the optic axis.

10. The accommodative lens system of claim 1 wherein the optic body of said first lens has a small hole through it.

11. The accommodative lens system of claim 10 wherein said small hole has a diameter in the range about 50 to about 100 μm.

12. The accommodative lens system of claim 11 wherein said first lens has a plate haptic body design.

13. The accommodative lens system of claim 12 wherein said plate haptic body has a thickness in the range of about 50 to about 200 μm, and wherein the overall length of said first lens is approximately 12 mm or less.

14. The accommodative lens system according to claim 2 wherein the radius of the posterior surface of said first lens is approximately same as the radius of the anterior surface of said second lens such that when said first lens shifts toward said second lens, said posterior surface of said first lens will be in substantial contact with said anterior surface of said second lens.

15. The accommodative lens system of claim 14 wherein said first lens is a plate haptic body design.

16. The accommodative lens system of claim 15 wherein said plate haptic body has a thickness in the range of about 50 to about 200 μm, and wherein the overall length of said first lens is approximately 12 mm or less.

17. The accommodative lens system of claim 2 wherein the optic body of said first lens has a small hole through it; and wherein the radius of the posterior surface of said first lens is approximately same as the radius of the anterior surface of said second lens such that when said first lens shifts toward said second lens, said posterior surface of said first lens will be in substantial contact with said anterior surface of said second lens.

18. The accommodative lens system of claim 17 wherein said small hole has a diameter in the range of about 50 to about 100 μm.

19. A method for implanting an accommodative lens system in the eye of a patient, comprising the steps of:
(a) making an incision in the eye;
(b) implanting through said incision a second lens with a positively powered optic body, implanted inside the capsular bag of the patient; and
(c) implanting through said incision a first lens with a negatively powered optic body, substantially in the posterior chamber of the eye, such that it is not fixed in a specific position in the eye, but rather remains in a central position in the pupil and is capable of shifting along the optic axis of the eye.

20. The method of claim 19 wherein said first lens is configured so as to permit the flow of aqueous humor past it, as said lens shifts positions in the eye.

21. The method of claim 20 wherein said first lens is a plate haptic body design.

22. The method of claim 21 wherein said plate haptic body has a thickness in the range of about 50 to about 200 μm, and wherein the overall length of said first lens is approximately 12 mm or less.

23. The method of claim 20 wherein said first lens is a multiple piece lens design wherein two or more haptic bodies are attached to and extend from the central optic body.

24. The method of claim 20 wherein the second lens is a full-sized IOL.

25. The method of claim 20 wherein the second lens is a plate haptic body design.

26. The method of claim 20 wherein said second lens is a multiple piece lens design wherein two or more haptic bodies are attached to and extend from the central optic body.

27. The method of claim 20 wherein said second lens is configured to allow its optic body to shift along the optic axis.

28. The method of claim 20 wherein the optic body of said first lens has a small hole in its center.

29. The method of claim 28 wherein said small hole in the range of about 50 to about 100 μm.

30. The method of claim 20 wherein the radius of the posterior surface of said first lens is approximately the same as the radius of the anterior surface of the second lens such that when said first lens will be in substantial contact with said anterior surface of said second lens.

31. The method of claim 30 wherein the optic body of said first lens has a small hole in its center, the diameter of said hole being from about 50 to about 100 μm.

32. A method for implanting an accommodative lens system in the eye of a patient retaining their natural crystalline lens, comprising the steps of:
(a) making an incision in the eye; and
(b) implanting through said incision a phakic lens with a negatively powered optic body, substantially in the posterior chamber of the eye, such that it is not fixed in a specific position in the eye, but rather remains in a central position in the pupil and is capable of shifting along the optic axis of the eye.

33. The method of claim 32 wherein phakic lens is configured so as to permit the flow of aqueous humor past it, as said lens shifts positions in the eye.

34. The method of claim 33 wherein said phakic lens has a small hole at the center of the optic body.

35. The method of claim 34 wherein said small hole has a diameter in the range of about 50 to about 100 μm.

36. The method of claim 33 wherein said phakic lens is a plate haptic body design.

37. The method of claim 36 wherein said plate haptic body has a thickness in the range of about 50 to about 200 μm, and wherein the overall length of said phakic lens is approximately 12 mm or less.

38. The method of claim 33 wherein said phakic lens is a multiple piece lens design wherein two or more haptic bodies are attached to and extended from the central optic body.
